(19) United States
(12) Patent Application Publication
(10) Pub. No.: US 2008/0262601 A1
(43) Pub. Date: Oct. 23, 2008

(54) Stent Device with Multiple Helix Construction

(76) Inventors: Edward H. Cully, Flagstaff, AZ (US); Michael J. Vonesh, Flagstaff, AZ (US)

Correspondence Address: David J. Johns;W. L. Gore & Associates, Inc., 551 paper Mill Road, P.O. Box 9206, Newark, DE, 19714-9206, US

(21) Appl. No.: 12/146,656

(22) Filed: Jun. 26, 2008

Related U.S. Application Data
(63) Continuation of application No. 10/242,999, filed on Sep. 13, 2002.

(51) Int. Cl. A61F 2/88

(52) U.S. Cl. 623/1.16

(57) ABSTRACT

An improved stent design is disclosed that employs a series of helically oriented expansion elements encircling the stent. Each of the expansion elements includes a stepped pattern employing two distinct pitch angles. The expansion elements are oriented to cooperate with each other to form a series of virtual radially expandable rings that provide suitable outward force for proper stent function, but which are not connected together to form a continuous coherent ring if separated from the stent as a whole. In this manner, a distinctive stent design is provided that has numerous functional benefits over stents described in the prior art.

### RELATED APPLICATION

This application is a continuation of co-pending application Ser. No. 10/242,999, filed Sep. 13, 2002.

### BACKGROUND OF THE INVENTION

1. Field of the Invention

The present invention relates to medical devices and more particularly to medical devices that are designed to be inserted endoluminally in a body.

2. Description of Related Art

Recent developments in medicine have emphasized minimally invasive surgical procedures. It is common today for medical instruments to be remotely inserted into a patient's body through small, sometimes percutaneous, incisions and entire operations performed remotely using fluoroscopic, radiographic, ultrasonic, angioscopic, or other visualization techniques.

These remote techniques are regularly employed today in a variety of vascular procedures, including treatments for coronary artery disease or other vascular obstructions (e.g., balloon angioplasty and/or stenting), repair of aortic or other vascular aneurysms, creation of various vascular shunts, repair of heart defects, correction of other duct problems in the body, etc. Despite tremendous advancements in the area of minimally invasive interventions, additional improvements are believed possible, and are likely necessary to fully exploit the potential of this technology.

Specifically, it is common today for expandable stent devices to be placed in a vessel to help maintain flow through the vessel or to prevent fluid from filling an aneurysm or from leaking through a tear or other opening in the vessel wall. Stents for these procedures may be formed from a plastically deformable material that is enlarged in place within the vessel (such as through use of an inflatable balloon), or through an elastic or springy material that allows the stent to self-expand in place once a constraint mechanism is removed from a compacted stent. In either case, the stent may include a covering on one or both of its inner or outer surfaces to prevent fluid flow from passing through the interstices of the stent and/or prevent cell ingrowth through the stent structure.

A wide variety of stent designs have been proposed to provide various beneficial properties. Many stents are formed from wire material that is wound and sometimes welded or otherwise joined into desired patterns. Alternatively, stents can be formed from continuous sheets or tubes that are then cut and formed into the desired stent pattern. Typically, both of these manufacturing techniques yield stent designs that fall into a couple basic forms.

A first common design for stents is to have an essentially helical design whereby a single stent element can be defined as extending helically around a longitudinal axis from one end of the stent to the other. Usually the helical stent element includes an undulating (e.g., “zigzag”) or other expandable pattern along its length. This design is particularly popular with wire-formed stents since it allows the stent to be formed from a single length of wire.

A second common design for stents is for the stent to comprise a series of discrete “ring” elements oriented essentially perpendicular to the longitudinal axis of the stent. The discrete ring elements are normally attached together by a series of one or more “connectors” or “bridges” extending between the rings. Again, the ring elements are usually formed with some form of undulating, diamond, serpentine, sinusoidal, or similar expandable pattern to allow compaction and/or expansion of the stent. By altering the shape and placement of the bridge elements it has been demonstrated that flexibility of the stent and its expansion properties can be tailored to address desired placement and operational specifications. Due to the complexity of many of the ring-and-bridge designs and the desire to avoid onerous forming and welding procedures, this design is most commonly employed with stents formed from a continuous tube or sheet of material that is cut into the desired pattern. A variation of this second type of stent is the so-called “closed-cell” design, typified by the J & J/Cordis Crown Stent and Medinol NIR stent.

While many of the existing stent designs function quite well for their intended purposes, it is believed that further improvements are possible. For example, with both of the above described common forms of stent designs it is often difficult to control the degree of shortening of the stent between its small delivery diameter and its enlarged deployed diameter. Generally for placement ease and the desire to minimize cell trauma, it is preferred to have minimal length change for the device while it is being enlarged in a vessel. Another common problem is that many existing stent designs are limited in their overall flexibility, making stent placement and expansion difficult or impossible in very small tortuous vessels.

It would be desirable to develop a stent that provides all the benefits of previous expandable stent devices while also having controlled shortening properties, excellent flexibility in the delivery and deployed configurations, and/or other desirable properties.

### SUMMARY OF THE INVENTION

The present invention comprises an improved stent for use in a variety of implantation procedures. The stent of the present invention comprises a series of radial expansion zones oriented essentially perpendicular to the longitudinal axis of the stent. Each of these radial expansion zones comprises at least two expansion elements that are not attached to or otherwise connected with each other within a defined radial expansion zone. Connection between the expansion elements can be provided outside of the radial expansion zones to provide overall stent continuity.

The present invention can be further defined as being a stent having multiple undulated expansion elements arranged around its longitudinal axis. Each of the expansion elements includes a first pitch angle oriented in a step-wise helical fashion around the longitudinal axis and a second pitch angle oriented essentially perpendicular to the longitudinal axis. By orienting the expansion elements relative to each other so that their second pitch angles are aligned with one another within a radial expansion zone, the expansion elements form a virtual radially expandable ring. However, unlike previous discrete ring stent devices, the expansion elements for the stent of the present invention are not connected to one another within the radial expansion zone(s). In this manner, the radial expansion elements are not independently radial expandable from each other.

The stent of the present invention provides a number of improved operating properties over previous stent designs. These include better longitudinal flexibility in both the compacted and expanded configurations, improved expansion characteristics, and controlled length change during expansion.

These and other benefits of the present invention will be appreciated from review of the following description.

### DESCRIPTION OF THE DRAWINGS

The operation of the present invention should become apparent from the following description when considered in conjunction with the accompanying drawings, in which:

FIG. 1 is a three-quarter perspective view of one embodiment of a stent of the present invention in its delivery (pre-expanded) configuration;

FIG. 2 is a three-quarter isometric view of the stent of FIG. 1 shown on a mandrel for clarity in visualizing its stent pattern;

FIG. 3 is a planar representation of the stent pattern of the pre-expanded stent of FIG. 1;

FIG. 4 is an enlarged side elevation view of the stent of FIG. 1;

FIG. 5 is a three-quarter isometric view of a transverse section of a single virtual radially expandable ring of the stent of the present invention cut along planes 48A and 48B of FIG. 4;

FIG. 6 is an exploded three-quarter isometric view of the virtual radially expandable ring of FIG. 5;

FIG. 7 is a side elevation view of the stent of FIG. 1;

FIG. 8 is a side elevation view of the stent of FIG. 1 shown in its fully expanded state;

FIG. 9 is a planar representation of the stent pattern of the fully expanded stent of FIG. 8;

FIG. 10 is a three-quarter isometric view of a stent of the present invention including a cover on its outer surface;

FIG. 11 is a three-quarter isometric view of a stent of the present invention including a coating thereon;

FIG. 12 is a planar representation in an unexpanded state of another embodiment of a stent pattern of the present invention employing a single helical element along its length;

FIG. 13 is a planar representation in an unexpanded state of another embodiment of a stent pattern of the present invention employing double helical elements along its length;

FIG. 14 is a planar representation in an unexpanded state of another embodiment of a stent pattern of the present invention employing quadruple helical elements along its length;

FIG. 15 is a planar representation in an unexpanded state of another embodiment of a stent pattern of the present invention employing quintuple helical elements along its length;

FIG. 16 is a planar representation in an unexpanded state of a further embodiment of a stent pattern of the present invention employing triple helical elements along its length and modified bridge members;

FIG. 17 is a planar representation in an unexpanded state of a further embodiment of a stent pattern of the present invention employing triple helical elements along its length and further modified bridge members;

FIG. 18 is a planar representation in an unexpanded state of a further embodiment of a stent pattern of the present invention employing triple helical elements along its length and further modified bridge members;

FIG. 19 is a planar representation in an unexpanded state of a further embodiment of a stent pattern of the present invention employing triple helical elements along its length and further modified bridge members;

FIG. 20 is a planar representation in an unexpanded state of a further embodiment of a stent pattern of the present invention employing triple helical elements along its length and bridge members that effectively form alternating radial expansion zones.

### DETAILED DESCRIPTION OF THE INVENTION

The present invention is an improved stent device for use in a variety of interventional procedures, such as treatments for coronary artery disease, or other vascular obstructions (e.g., balloon angioplasty and/or stenting), repair of aortic or other vascular aneurysms, creation of various vascular shunts, repair of heart defects, correction of other duct problems in the body, etc. As the term “stent” is used herein, it refers to a device that is adapted to be inserted into a vessel or other passageway or opening within a body and then deployed in place to assist in structurally supporting the host vessel lumen, maintaining patency through the vessel, passageway or opening, and/or to prevent liquids, cells, or other substances from passing through the side wall of the stent, particularly when used with a cover. A stent made in accordance with the present invention may be formed from either plastically deformable material that is expanded in place using a balloon or similar device, or an elastic or springy material that will self-expand in place following placement. Likewise, the stent of the present invention may also be configured to be a permanent implant or erode/resorb over time, incorporate various coatings resulting in a composite structure, and/or comprise a substrate for elution of drugs.

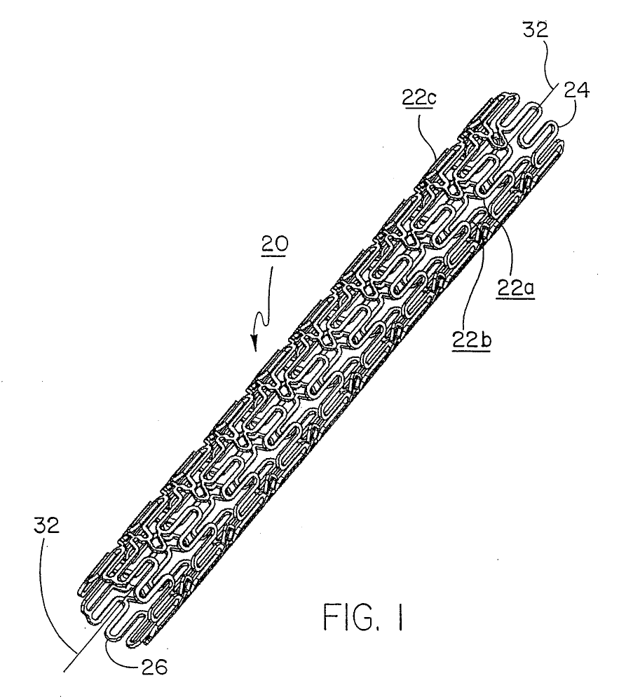

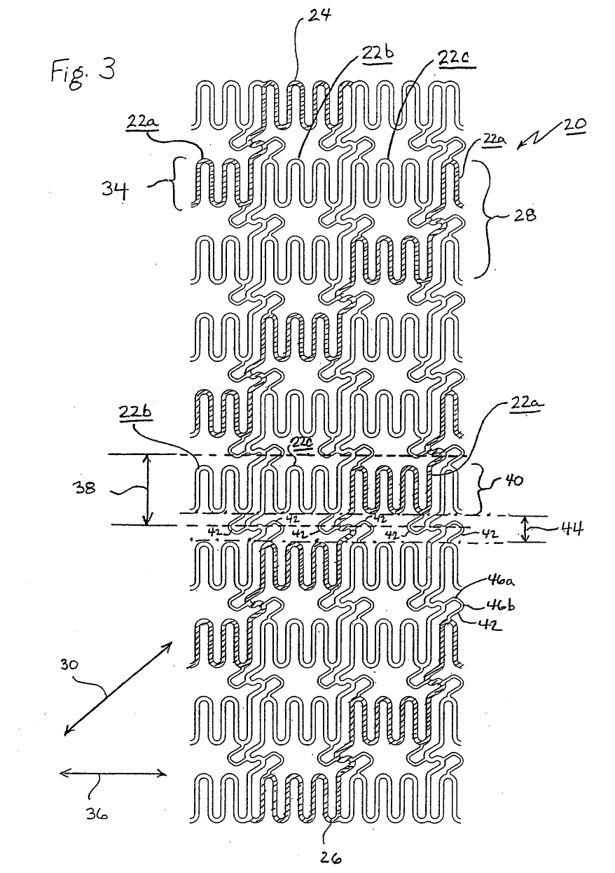

FIGS. 1 through 8 illustrate one embodiment of a stent 20 of the present invention. In this embodiment the stent 20 is formed from a continuous tube of material that is cut into the desired stent pattern. The stent pattern is one that is a hybrid of previous helical wire patterns and joined-ring patterns. The pattern, best seen in the two-dimensional view of FIG. 3, can be defined as having a series of helically disposed expansion elements 22a, 22b, 22c, that each extend from a first end 24 of the stent to a second end 26. Expansion element 22a has been cross-hatched for clarity. In this embodiment each of the expansion elements comprises an undulating pattern, although it is appreciated that any pattern enabling circumferential expansion is feasible with the concepts embodied in the present invention.

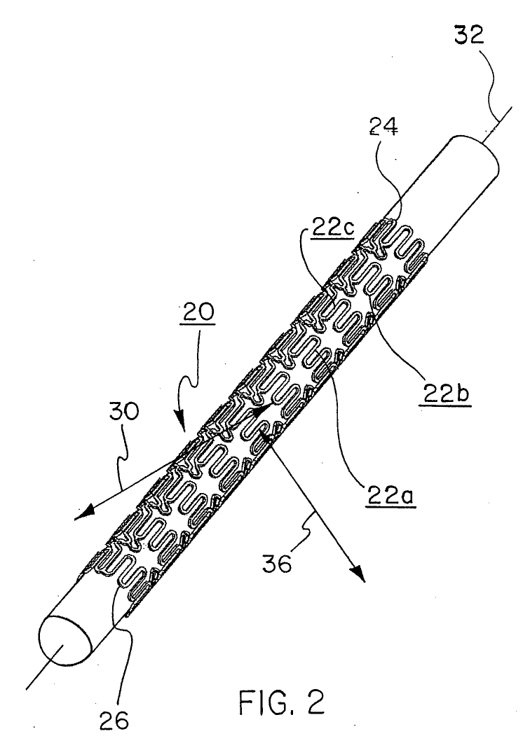

Focusing on only expansion element 22a in FIGS. 2 and 3, the expansion element 22a takes on a stepped orientation having segments 28 with a first pitch angle 30 extending helically around a longitudinal axis 32 of the stent 20 and segments 34 with a second pitch angle 36 positioned approximately perpendicular to the longitudinal axis 32.

As is shown in FIG. 3, by aligning the second pitch angles of each of the expansion elements 22a, 22b, 22c together in a radial expansion zone 38 the multiple expansion elements cooperate to form a virtual radially expandable ring 40. However, unlike previous joined-ring stent designs which are circumferentially continuous, and thus provide a closed cylindrical structure, the expansion elements of the present invention are not attached to each other within the radial expansion zone 38. In this manner, within the radial expansion zone the three expansion elements are separated from each other (i.e., non-continuous), and none of the expansion elements are independently radially expandable (that is, a central radial expansion force applied to the stent within a radial expansion zone will necessarily expand each of the separate expansion elements within the zone at the same time and it is not feasible to expand any one of the expansion elements independently from its adjacent elements). Likewise, the radial expansion zone does not comprise a closed cylindrical structure. This unique feature imparts enhanced flexibility to the present invention in both the expanded and non-expanded configurations.

Connecting bridges 42 are provided in connection zones 44 positioned between (and possibly overlapping) the radial expansion zones 38. The bridges 42 may be constructed to include one or more bends 46a, 46b or other means to provide stored-length therein. The stored-length of the bridges allows the stent to expand radially while not significantly foreshortening in the expansion process. Similarly the bridges can be used to alter the flexural modulus of the stent as well as the degree of endoluminal scaffolding.

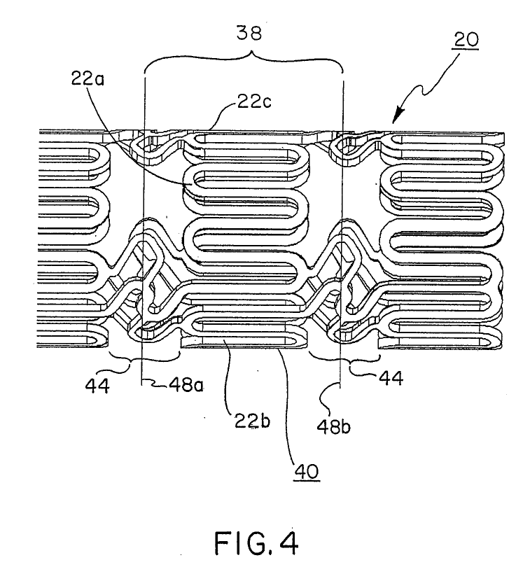

The construction and function of the expansion elements within the radial expansion zone can be better appreciated through review of FIGS. 4 through 6. FIG. 4 illustrates an enlarged view of a radial expansion zone 38 of the present invention comprising expansion elements 22a, 22b, 22c. The radial expansion zone 38 is defined by establishing two sectioning planes 48a, 48b through the mid-point of the connection zones 44 in a manner to establish a virtual radially expandable ring 40 that is symmetrical with adjacent virtual radially expandable rings in the stent 20. In other words, the radial expansion zone is sectioned so as not to encroach onto adjacent virtual radially expandable ring structures. In this manner, pairs of sectioning planes can be periodically applied along the entire length of the device to define the constituent virtual radially expandable rings forming the tubular stent.

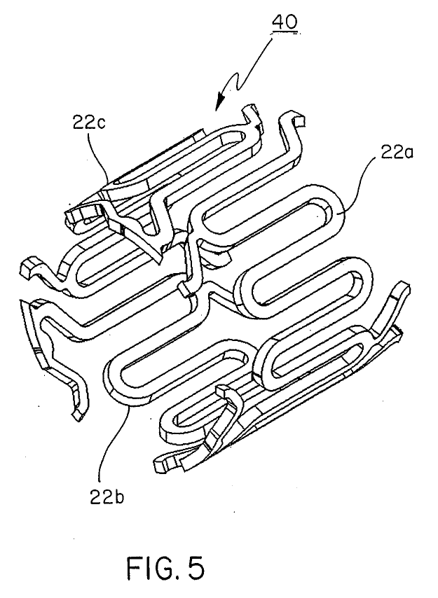

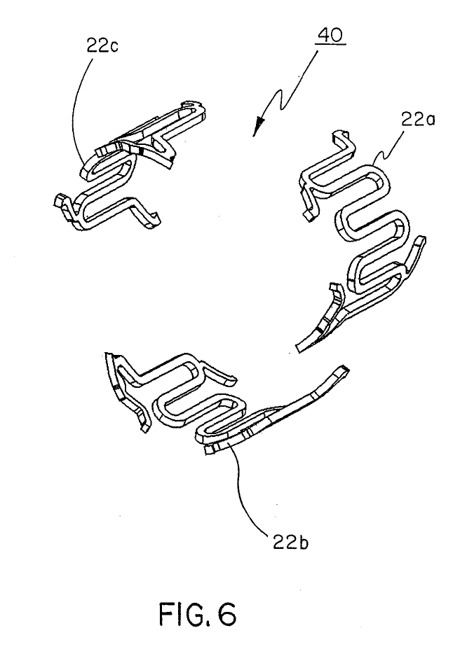

When the radial expansion zone 38 is formed in this manner, the zone 40 can be removed from the rest of the stent 20 structure to form a virtual radially expandable ring 40 as is shown in FIG. 5. While this virtual radially expandable ring 40 provides excellent radial force to hold open vessel walls and the like, none of the expansion elements 22a, 22b, 22c in the ring are actually connected together. The lack of interconnectedness among the expansion elements 22 within the radial expansion zone is shown in the exploded view of FIG. 6.

It is believed that there are a number of advantages to maintaining separate expansion elements within the radial expansion zone. First, the separation of these elements is believed to contribute to improved stent flexibility in the expanded and non-expanded configurations by facilitating independent movement of the expansion elements within the virtual ring structures. Second, it is believed that the separation of the expansion elements provides more consistent and predictable expansion properties along the entire length of the stent. Third, when combined with the appropriate bridge structures, this design provides for exact engineering of stent foreshortening properties.

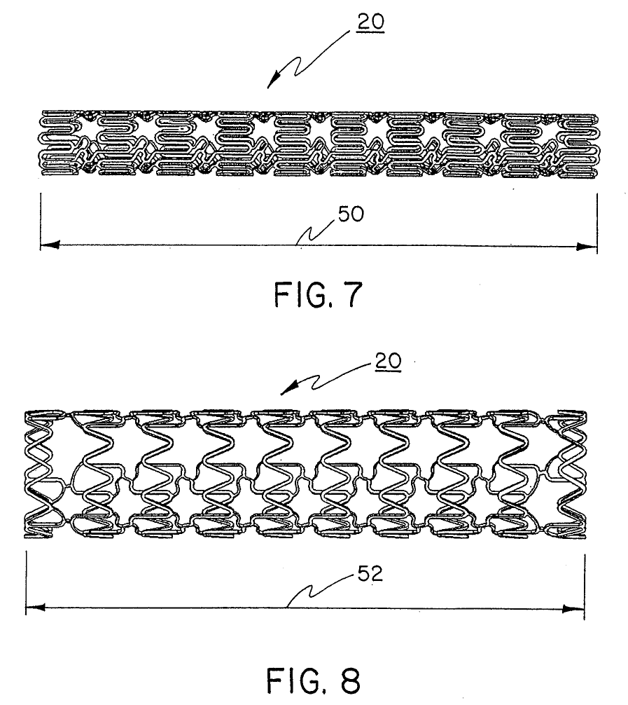

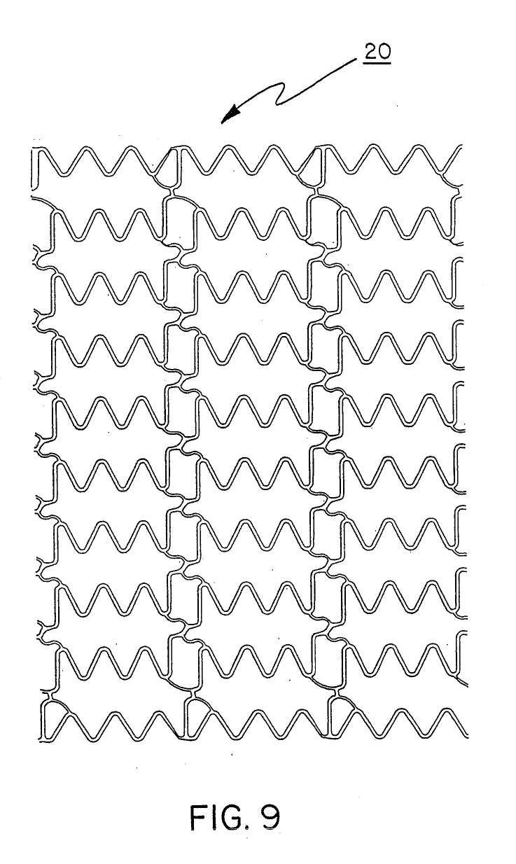

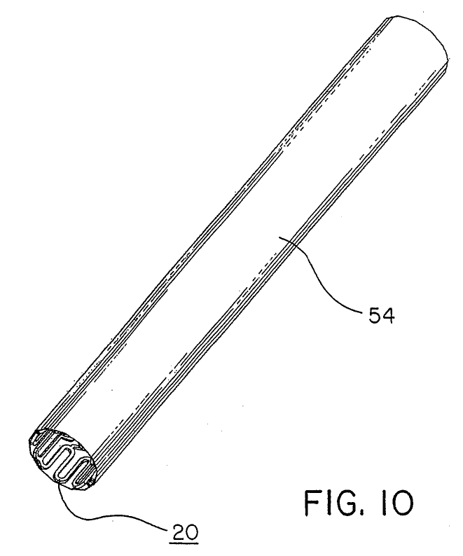

FIGS. 7 and 8 demonstrate how the stent 20 of the present invention expands from the compacted orientation of FIG. 7 to the fully enlarged orientation of FIG. 8 with minimal foreshortening of the stent along its length. As can be seen, the compacted stent 20 in FIG. 7 has a length 50 that is essentially the same as length 52 of the expanded stent 20 in FIG. 8. A two-dimensional representation of the stent 20 as expanded in FIG. 8 is illustrated in FIG. 9.

Maintaining a consistent overall length of the stent throughout expansion is highly desirable in order to make placement and deployment of the stent more accurate for the medical staff. Additionally, in order to minimize cell irritation or damage during deployment, it is also desirable not to have the stent moving longitudinally during the deployment process. The design of the present invention provides a wide choice of engineering options with respect to stent length change during deployment. In addition to allowing the stent 20 to undergo little or no change in length during deployment, with the design of the present invention it has been determined that by modifying the shape of the expansion elements and the bridges, the stent can be engineered to undergo anything from controlled shortening to even controlled lengthening during expansion.

The stent 20 of the present invention may be formed from a wide variety of materials, including metals (e.g., stainless steel or nitinol), plastics (e.g., PTFE or other fluoropolymers), resorbable materials (e.g., polymers or copolymers possessing one or more of the following monomeric components: glycolide (glycolic acid); lactide (d-lactide, l-lactide, d,l-lactide); trimethylene carbonate; p-dioxanone; caprolactone, hydroxybutyrate, hydroxyvalerate), any other material suitable for implantation, or combinations of any of these or other materials. Additionally, the stent may be provided with additional treatment or therapeutic agents, such a drugs, radiation, radiopaque markers or coatings, or other agents to enhance visualization in-vivo.

To construct the stent of the present invention it is preferred that the stent be cut from a continuous tube of material into the desired pattern, such as through use of a laser. The stent may also be constructed by machining, chemical etching, or other suitable means. The stent may also be formed from a flat sheet of material that is cut into the desired pattern and then bonded together to form a tube having a seam. Finally, although not preferred, the stent of the present invention may be constructed from wires or ribbons that are formed into the desired shapes and then bonded together into the final pattern.

Stents of the present invention can be constructed in a variety of sizes and shapes, including compacted insertion diameters from less than 1 mm to more than 10 mm, and deployed diameters of less than 3 mm to more than 30 mm. It may also be desirable to form stents of the present invention that have tapered or stepped diameters along its length. Stents of the present invention also may be joined together, such as to form a bifurcated stent device, or stent device with a side branch.

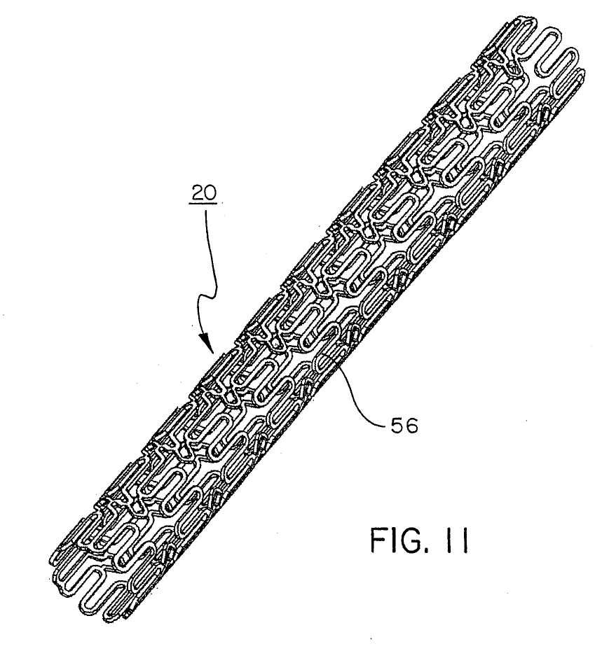

In instances where the stent of the present invention is used to isolate cells, aneurysms, vessel wall defects, and the like, it may be desirable to provide a cover 54 on the stent 20, as is shown in FIG. 10. Suitable cover materials include polytetrafluoroethylene (PTFE), expanded PTFE, other fluoropolymers such as fluorinated ethylene propylene (FEP), polyethylene, polypropylene, fluoroelastomer, or a resorbable material. Such covers may be mounted on the stent on its inside, outside, or both over all or a portion of the device length. Additionally, a cover may be provided that allows the stent to be embedded within the cover material, such as through use of a silicone or other elastomeric material. Covers may be coextensive with the length of the stent, as is shown in FIG. 10, or they may be either longer or shorter than then stent. Additionally, multiple stents may be provided within a single cover material or multiple covers may be joined together using a stent of the present invention. Further, one or more openings may be provided in the cover material along its length, for instance to accommodate communication with side vessels or similar applications. The cover may be attached to the stent in any suitable manner, including adhesive, friction fit, tape or other tacking material, heat or other bonding techniques, etc. It should be evident from this description that the stent of the present invention may be used with cover materials in any manner now known or later developed without departing from the present invention.

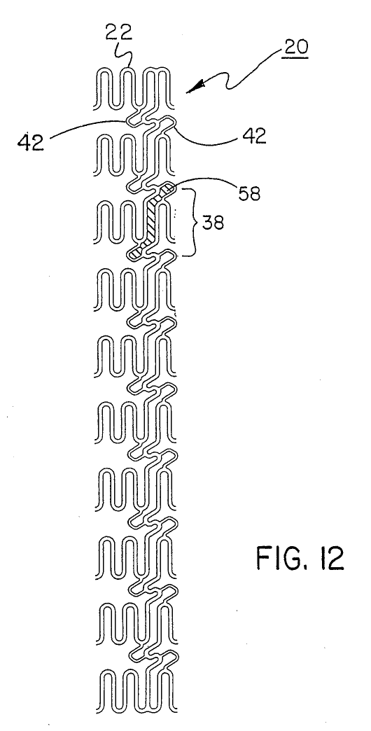

Instead of or in addition to a cover material, the stent of the present invention may include a coating 56 on its surface, as is illustrated in FIG. 11. Suitable coating materials may include: fluoroelastomer, ceramic, silicone, polyethylene, carbon, gold, Heparin, hydrogel, or lubricious coatings. Coating materials can provide numerous benefits, including protecting the underlying stent material, providing a substrate for delivery of drugs or other therapeutic substances, isolating the stent material from interaction with surrounding cells, improving fluoroscopic visualization. Coatings can be applied in any material-appropriate manner, such as dip-coating, spray-coating, electro-deposit, or chemical vapor deposition. Additionally, depending upon the application, coatings can be provided to all or only some of the stent surface. Again, coatings can be applied to the stent of the present invention in any form now known or later devised.

Without departing from the present invention it is possible to modify its stent pattern to provide different stent dimension and/or different stent performance properties. Some of the many permutations of stent designs within the scope of the present invention are illustrated in FIGS. 12 through 20. Each of these embodiments share the common feature of a repeating series of radial expansion zones 38 arranged along a common longitudinal axis.

FIG. 12 illustrates a stent 20 of the present invention that employs a single helical expansion element 22 along its length. The use of bridges 42 in this embodiment attaches the expansion element to itself. As can be seen, this embodiment provides a single expansion cell 58 per radial expansion zone 38. This embodiment is particularly suitable for stents for extremely small diameter applications and/or applications requiring extreme longitudinal flexibility.

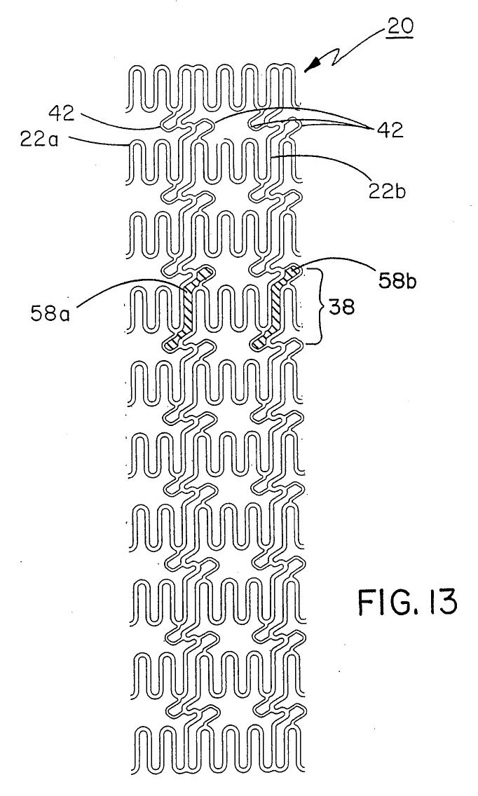

FIG. 13 illustrates a stent 20 of the present invention that employs a pair of helical expansion elements 22a, 22b along its length. The bridges 42 join expansion element 22a to expansion element. 22b. In this form a pair of expansion cells 58a, 58b is provided per radial expansion zone 38.

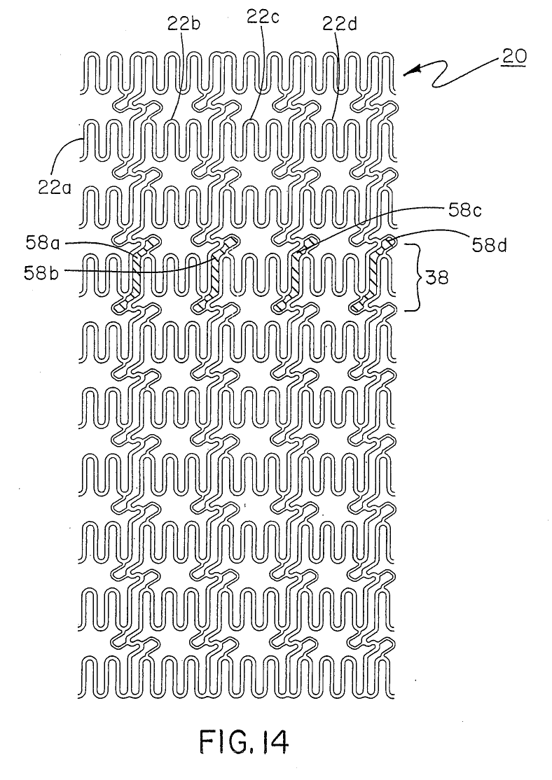

FIG. 14 illustrates a larger diameter stent 20 having four expansion elements 22a, 22b, 22c, 22d along its length. This provides four sets of expansion cells 58a, 58b, 58c, 58d per radial expansion zone 38.

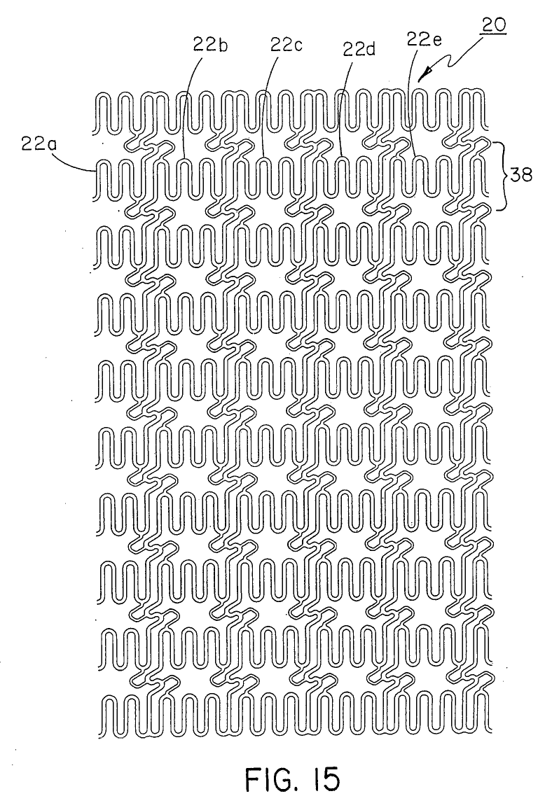

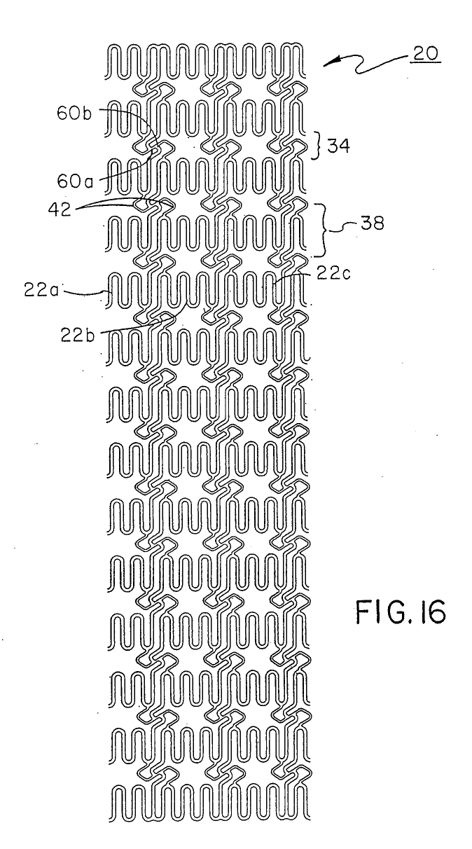

For particularly large diameter applications, FIG. 15 illustrates a stent 20 of the present invention that employs five expansion elements 22a, 22b, 22c, 22d, 22e along its length. It should be evident from this description that the number of expansion elements 22 may be increased or decreased to any appropriate number to provide suitable stent performance characteristics.

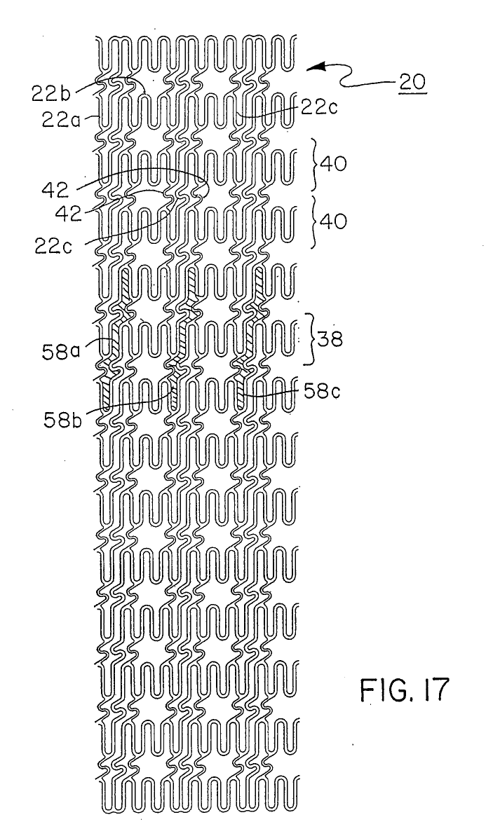

FIG. 16 illustrates a stent 20 of the present invention that employs three expansion elements 22a, 22b, 22c with modified bridge 42 structures within the connection zone 34. In this embodiment the bridges 42 do not attach to the expansion elements 22 at a single point, but, rather, attach to the expansion element at two separate attachment points 60a, 60b.

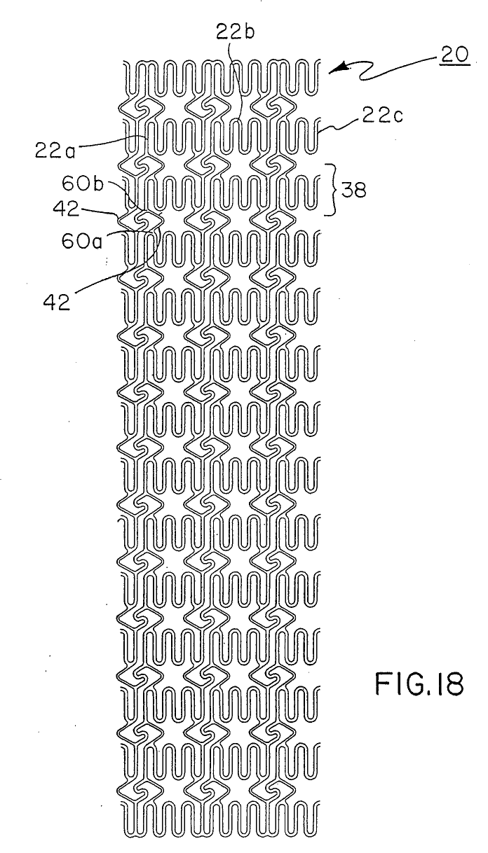

FIG. 17 illustrates a stent 20 of the present invention that employs three expansion elements 22a, 22b, 22c with further modified bridge 42 structures within the connection zone 34. In this embodiment the bridges 42 do not connect to the expansion element that passes through connection zone 34, as in the previously described embodiments. Instead, the bridges 42 of this embodiment connect between longitudinally adjacent expansion elements 22 that form the virtual radially expandable ring 40. The stent pattern of this embodiment creates particularly large expansion cells 58a, 58b, 58c that extend over multiple radial expansion zones 38.

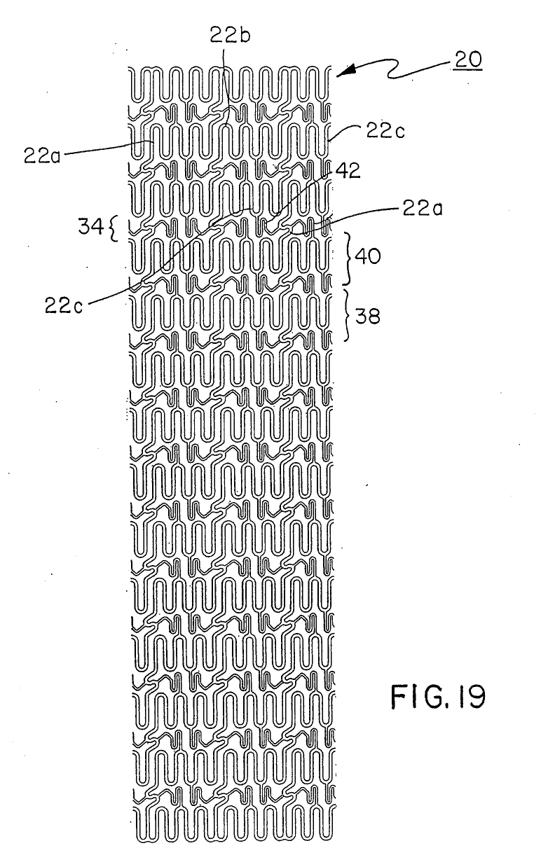

FIG. 18 illustrates a stent 20 of the present invention that employs three expansion elements 22a, 22b, 22c and further modified bridge 42 structures. Like the embodiment illustrated in FIG. 16, the bridges 42 of this embodiment attach to the expansion elements at separate points 60a, 60b, but are positioned at a greater distance from each other.

FIG. 19 illustrates a stent 20 of the present invention that employs three expansion elements 22a, 22b, 22c and still further modified bridge 42 structures. The bridges 42 in this embodiment attach between the expansion elements 22 (for example, expansion element 22a as designated in the Figure) that pass through connection zone 34 and the expansion elements 22 (for example, expansion element 22c as designated in the Figure) that form the virtual radially expandable ring 40.

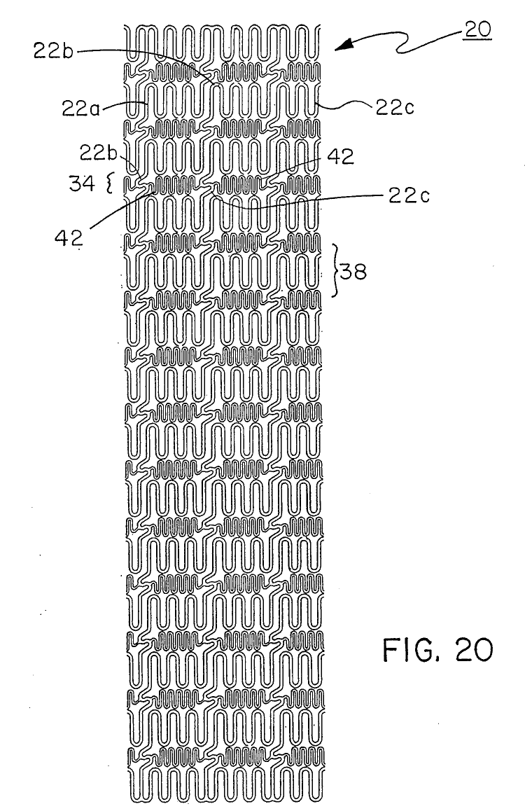

FIG. 20 illustrates a stent 20 of the present invention that employs three expansion elements 22a, 22b, 22c and another modification of the bridges 42 structures. The bridges 42 in this embodiment attach directly between the expansion elements 22 that pass through connection zone 34 (for example, between expansion elements 22b and 22c as designated in the Figure). Formed in this manner, the bridges 42 effectively form alternating radial expansion zones with the expansion elements 22.

While particular embodiments of the present invention have been illustrated and described herein, the present invention should not be limited to such illustrations and descriptions. It should be apparent that changes and modifications may be incorporated and embodied as part of the present invention within the scope of the following claims.

The invention claimed is:

1. A stent having a longitudinal axis comprising
multiple undulated elements arranged around the longitudinal axis, each undulating element including a first pitch angle oriented helically around the longitudinal axis and a second pitch angle oriented essentially perpendicular to the longitudinal axis;
wherein the undulating elements are oriented relative to each other so that their second pitch angles are aligned with one another within a radial expansion zone; and
wherein the undulating elements are not connected to one another within the radial expansion zone.

2. The stent of claim 1 wherein each undulating element is connected to an adjacent undulating element.

3. The stent of claim 1 wherein the stent includes
a series of radial expansion zones oriented essentially perpendicular to the longitudinal axis; and
within each radial expansion zone there are at least two separate undulating elements.

4. The stent of claim 3 wherein within each radial expansion zone there are at least three separate undulating elements.

5. The stent of claim 1 wherein when the radial expansion zone is separated from the stent as a whole, each of the undulating elements in the expansion zone will readily separate from one another.

6. The stent of claim 1 wherein the undulating elements in the radial expansion zone are not independently radial expandable from each other.
